(19) United States
(12) Patent Application Publication
(10) Pub. No.: US 2025/0256107 A1
(43) Pub. Date: Aug. 14, 2025

(54) PACEMAKER AND OPERATION METHOD OF SUCH PACEMAKER

(71) Applicant: BIOTRONIK SE & Co. KG, Berlin (DE)

(72) Inventors: Kurt SWENSON, Corvallis, OR (US); Brian M. TAFF, Portland, OR (US); Hannes KRAETSCHMER, West Linn, OR (US); Karl-Heinz FREIBERG, Woodburn, OR (US); David MILLER, Lake Oswego, OR (US); Madeline Anne MIDGETT, Portland, OR (US); Iris SHELLY, Portland, OR (US); Daniel YOUNG, Portland, OR (US); R. Hollis WHITTINGTON, Portland, OR (US); Brad MCMILLAN, Lake Oswego, OR (US)

(73) Assignee: BIOTRONIK SE & Co. KG, Berlin (DE)

(21) Appl. No.: 18/857,585

(22) PCT Filed: Apr. 4, 2023
(86) PCT No.: PCT/EP2023/058803

§ 371 Date: Oct. 17, 2024

Related U.S. Application Data
(60) Provisional application No. 63/335,329, filed on Apr. 27, 2022.

(30) Foreign Application Priority Data
May 20, 2022 (EP) 22174488.1

(51) Int. Cl. A61N 1/365; A61N 1/02; G16H 40/63

(52) U.S. Cl. CPC A61N 1/36592; A61N 1/025; A61N 1/36507; G16H 40/63

(57) ABSTRACT

Cardiac pacemaker for a patient's heart including a processing unit with a data memory, further including a first detector, and a second detector, a pacing signal generator, which are all electrically connected to the processing unit, wherein the first detector is configured to detect time-dependent electrical signals of the heart, wherein the second detector is configured to detect time-dependent bodily signals of the patient different from the signals detected by the first detector, wherein the first detector and the second detector are configured to transmit the detected and, if applicable, pre-processed signals to the processing unit, wherein the processing unit is configured to process the signals received from the first detector and the signals from the second detector and to detect from the received signals of the first detector intrinsic atrial events, intrinsic ventricular events and pacing events and to determine an actual cardiac rate.

### CROSS-REFERENCE TO RELATED APPLICATIONS

This application is the United States National Phase under 35 U.S.C. § 371 of PCT International Patent Application No. PCT/EP2023/058803, filed on Apr. 4, 2023, which claims the benefit of European Patent Application No. 22174488.1, filed on May 20, 2022 and U.S. Provisional Patent Application No. 63/335,329, filed on Apr. 27, 2022, the disclosures of which are hereby incorporated by reference herein in their entireties.

### TECHNICAL FIELD

The present invention is generally directed to a cardiac pacemaker and an operation method of such pacemaker, a respective computer program product and computer readable data carrier.

### BACKGROUND

A cardiac pacemaker (or artificial pacemaker) is a medical device that generates electrical pulses delivered by electrodes connected to or fixed at the pacemaker to cause the heart muscle chambers (i.e., the atria and/or the ventricles) to contract and therefore pump blood. By doing so this device replaces and/or regulates the function of the electrical conduction system of the heart. One purpose of a pacemaker is to maintain an adequate heart rate (cardiac rate), either because the heart's natural pacemaker is not fast enough, or because there is a block in the heart's electrical conduction system. Additionally, or alternatively, the pacemaker may stimulate different positions within the ventricles to improve the synchronization of the ventricles or provide anti-tachycardia operations to treat life-threatening arrhythmias. Modern pacemakers are externally programmable and allow a health care professional (HCP) to select the optimal pacing mode(s) for individual patients.

A conventional pacemaker comprises a controlling and generator device comprising a processing unit and a power source external of the patient's heart and electrodes that are implanted within the heart's muscle. The electrodes are connected via leads and a header located on device. In most cases the device is implanted subcutaneously in the front left or right side of the chest. An implantable intra-cardiac pacemaker (also known as implantable leadless pacemaker-ILP) is a miniaturized pacemaker which is entirely implanted within a heart's ventricle (V) or atrium (A) of a patient. ILPs are considered the future of cardiac pacing. Alternative or additional functions of conventional or intra-cardiac pacemakers comprise providing other electrical or electromagnetic signals to the heart or its surrounding tissue and sensing electrical or electromagnetic signals (e.g., signals from electrical depolarization fields) or other physiological parameters of the heart and/or its surrounding tissue such as the intrinsic (i.e., the heart's natural) atrial contraction or the intrinsic (i.e., the heart's natural) ventricular contraction. Due to the highly restricted device size, an ILP has a small battery capacity.

The pacing functionality of a conventional pacemaker or ILP is aimed at staying synchronous, as far as possible, with the heart's natural activity.

Additionally, pacemakers are known to use specific programmed rates for administering paced output therapies to the heart. Those specific rates may be realized by counting a clock signal and referencing it against intervals corresponding to the said specific rates. The simplest pacemakers use a fixed, typically programmable, rate which meets the needs of the patient under most circumstances. Using a sensor-derived rate that is based on a demand-correlated measurement, such as acceleration, is common. For replacing failed AV conduction signals, AV synchronous pacemakers attempt to pace the ventricle at a rate that corresponds with a detected atrial event (e.g., atrial contraction event or atrial depolarization event). It is also possible to deduce a physiologically missing electrical heart rate signal from other systems in the body (e.g., the brain and baroreceptors) by leveraging inputs from other sensor types.

An inherent rate used by all modern pacemakers is the intrinsic rate of the heart. When the intrinsic rate is near the pacing rate, the refractory periods of the cardiac conduction paths tend to block the conduction of the intrinsic electrical signals, but when the intrinsic signal is at a rate that is significantly faster than the pacing rate, the signals may conduct and trigger heart chamber contractions. These are sensed by the pacemaker and such sense detections are used to inhibit pacing.

Leadless pacemakers largely aim to provide support as has been delivered for bradycardia management in traditional, pocket based leaded IPGs through devices sized at ˜10% of the total volume of legacy formats. While this miniaturization permits the placement of leadless implants within the blood volume of a patient's heart and provides reduced risks for regurgitation and infection through the elimination of leaded interfacing with the myocardium, the approach eliminates the faculties for directly measuring cardiac signaling emergent from a multitude of heart chambers (barring configurations that leverage wireless networking with other separate implants) as individual leadless pacers reside wholly within single heart chambers. With this changed design paradigm, there is therefore a need (again, in non-networked leadless pacemaker systems) for the implant to collect heart chamber information readily available to leaded systems “at a distance” in leadless embodiments in cases where synchronous mode support is desired. This capability demands that the affiliate mode support architecture, in turn, be one that offers effective means for managing behaviors subject to a reduced reliability in the quality of signaling and event detection emergent from chambers other than the one where the implant resides.

One presently available right-ventricle-stationed leadless pacing system offering AV synchronous therapy output gathers its “at a distance” atrial event signaling using mechanical inputs from the in-device accelerometer. By using mechanical inputs to assess atrial signaling, the device's VDD-style mode support necessarily demands that the implant both simultaneously run its mode management timer model while also keeping the accelerometer in an ON state. In addition to this mechanical input configuration offering limited fidelity in assessing heart events within the atrium, such methods impose limits on the maximum accessible upper tracking rates due to the inherent delays associated with lapses between electrical depolarization effects and the resultant mechanical contraction within a heart chamber. Further, the current consumption necessary to run the timer model management and the on-board accelerometer at the same time elevates the device's baseline current consumption substantially (from 1.3 μA to 1.6 μA), threatening device longevity for an implant where change-out/follow-on therapy support is a complicated and arguably contentious affair.

Accordingly, there is the need for a cardiac pacemaker that copes with the unreliable detection of intrinsic atrial events and administers output in accordance with the patient's changing metabolic needs. Additionally, especially in the case of an ILP, the pacemaker must meet challenging bradycardia symptom management support needs subject to a power budget severely limited relative to legacy device designs.

The present disclosure is directed toward overcoming one or more of the above-mentioned problems, though not necessarily limited to embodiments that do.

### SUMMARY

At least the above problem is solved by a cardiac pacemaker comprising the features of claim 1, an operation method of a cardiac pacemaker with the features of claim 7, a computer program product with the features of claim 14 and computer readable data carrier having the features of claim 15.

In particular, at least the above problem is solved by a cardiac pacemaker for a patient's heart comprising a processing unit with a data memory, further comprising a first detector, and at least one second detector, a pacing signal generator, which are all electrically connected to the processing unit, wherein the first detector is configured to detect time-dependent electrical signals of the heart, for example, an intra-cardiac electrogram, wherein the at least one second detector is configured to detect time-dependent bodily signals of the patient different from the signals detected by the first detector, wherein the first detector and the at least one second detector are configured to transmit the detected and, if applicable, pre-processed signals to the processing unit, wherein the processing unit is configured to process the signals received from the first detector and the signals from the at least one second detector and to detect from the received signals of the first detector intrinsic atrial events, intrinsic ventricular events and pacing events (and/or understand the sequencing of pacing events) and to determine an actual cardiac rate, wherein the processing unit comprises a state determining module, wherein the state determining module is configured to dynamically select one of at least one first state, at least one second state and a third state and to use the selected state to determine the ventricular pacing time and/or rate information to meet the patient's actual or therapeutic needs,
wherein in the at least one first state the actual cardiac rate is determined from the detected atrial intrinsic events, and/or ventricular intrinsic events and/or pacing events and the current ventricular pacing time and/or rate information is determined in a VDD mode with atrial tracking based on the determined actual cardiac rate,
wherein in the at least one second state the determination of the actual cardiac rate from the detected atrial intrinsic events and ventricular intrinsic events is prohibited and the ventricular pacing time and/or rate information is determined based at least partly on the signals received from the at least one second detector and/or based on a first pre-defined pacing rate,
wherein in the third state the actual cardiac rate is determined from the detected atrial intrinsic events, ventricular intrinsic events and/or pacing events and the ventricular pacing time and/or rate information is determined based on a second pre-defined pacing rate or the determined actual cardiac rate dependent on the up-to-dateness of the actual cardiac rate,
wherein the state determining module is configured to switch from one state to another state of said states (i.e., the at least one first state, the at least one second state and the third state), wherein switching from the at least one first state directly to the third state is prohibited, depending on the present state and on at least one of a set of pre-defined conditions.

The switching may include the switching from at least one first state to the at least one second state, from the at least one second state to the third state and from one first state to another first state and from one second state to another second state.

In one embodiment, the processing unit is configured to process the signals received from the first detector and the signals from the at least one second detector and to detect from the received signals of the first detector intrinsic atrial events, intrinsic ventricular events and pacing events and to determine the actual cardiac rate together with an information on an up-to-dateness of the actual cardiac rate, wherein the up-to-dateness determination is based on the detected intrinsic atrial events and intrinsic ventricular events, wherein the processing unit is configured to determine and transmit a ventricular pacing time and/or rate information to the pacing signal generator for providing a pacing signal for the patient's heart based on this information.

An actual cardiac rate may also be called as a measured intrinsic cardiac rate and vice versa.

The key points of the descriptions above may be: we have two detectors—one for cardiac electrical signals and another for patient body/metabolic need; we use a processor in the implant to take information from the 2 detectors to facilitate therapy using three timer sub-states; sub-state one is an atrial tracking state that uses the 1st detector; sub-state two is a non-tracking state that may or may not leverage key inputs from the 2nd detector while also not necessarily excluding all inputs from the 1st detector; sub-state three is a state that attempts to re-establish tracking by initiating AV delays subject to detected atrial events.

In the at least one first state, the state determining module uses VDD pacing mode. The VDD pacing mode has a behavior in which the ventricle is stimulated according to the intrinsic atrial event and presence or absence of an intrinsic ventricular event. This means that pacing of the ventricle is inhibited if an intrinsic ventricular event is detected within a specified interval (the AV interval) or pacing of the ventricle is triggered if an intrinsic ventricular event is not detected within that specified interval. The interval may be prolonged using a pre-defined hysteresis. In the at least one first state the state determining module uses atrial tracking which means that the start of the AV interval is adapted to the detected intrinsic atrial event of the respective cycle if this event is detected within a pre-defined time interval.

The at least one second state and the third state may be regarded as states realizing VDD mode sub-states. In one embodiment, the state determining module is configured such that ventricular pacing time and/or rate information is determined using a VVI-behavior or a VVI-R behavior in the at least one second state as explained below in more detail. The VVI behavior comprises a pacing that does not consider atrial activity but is based on a pre-defined pacing rate. The VVI-R behavior provides a pacing rate dependent on the signal of the second detector representing the actual activity of the patient. In the second state, tracking of atrial and/or ventricular events (including intrinsic events and pacing events) is turned off as the actual cardiac rate is not determined from these events. This behavior may cause reduction in power consumption.

The third state may be regarded as a transition state which is used by the state determining module to re-synchronize the intrinsic atrial and ventricular events and the pacing to return to the at least one first state. At the beginning, the actual cardiac rate may be determined based on a second pre-defined pacing rate stored in the data memory, e.g., a resting rate of the patient. The third state may use the first detector and may detect atrial and/or ventricular intrinsic events/senses. If an atrial intrinsic event is detected, atrial tracking may be used, wherein an AV delay derived from the actual cardiac rate may be started from this event to phase-shift the pacing thereby providing re-synchronization of the pacing with the intrinsic events of the patient's heart. Further detection of atrial and/or ventricular intrinsic events may be used to determine the actual or measured intrinsic cardiac rate and increase its up-to-dateness (also referred to as freshness as is explained in further detail below). If the pacing is in correct phase and the actual or measured intrinsic cardiac rate has sufficient up-to-dateness (freshness), the state determining module is configured to switch to the at least one first state. In other words, the at least one first state is any atrial tracking state, the at least one second state is a non-atrial-tracking state which does not use any intrinsic atrial or ventricular event information to initiate AV delays, and the third state is a resynchronization state where any available atrial information is used to initiate an AV delay but the conditions are not yet sufficient to achieve consistent atrial tracking. The state determining module switches in the above-described manner between these states to meet the patient's needs in pacing.

In one embodiment, the set of pre-defined conditions comprises at least two of the following conditions:
the up-to-dateness of the actual or measured intrinsic cardiac rate,
a comparison of the actual cardiac rate and a pre-defined upper rate threshold or a pre-defined lower rate threshold,
the signals received from the at least one second detector, and
a comparison of a time elapsed from a pre-defined time point in connection with the switch to the present state and a pre-defined time interval threshold.

Rate thresholds (also referred to as check rates) as indicated above may trigger an interim or transition state where analysis of patient/device interactions may serve to determine how to proceed. One example of this type of check includes a supraventricular tachycardias (SVT) suspicion support. Here, triggered by the rate threshold, the state determining module may determine whether or not atrial event inputs should be utilized further for managing therapy output or whether leveraging other inputs (such as the motion sensor or a baseline low rate) should drive pacemaker behavior (notionally avoiding any tracking of problematic arrhythmia conditions).

The embodied system is not always capable of determining what is going on in the patient's cardiac system. In such cases the state determining module may provide a timing pattern that, while not necessarily optimum, is considered sufficient for all conditions by using, for example, a first pre-defined pacing rate in the at least one second state. Further, a timeout may be established to allow the patient/device system to settle into a more common condition, for example, a switch from the at least one second state to another second or the third state or a switch from the third state to the at least one first state. Accordingly, the state determining module may compare a time elapsed from a pre-defined time point in connection with the switch to the present state and a pre-defined time interval threshold (timeout). When the timeout is reached, this embodiment moves to the sub-state that encourages acquisition of intrinsic timing information to attempt to re-establish AV synchrony.

In one embodiment, the up-to-dateness (or lack of up-to-dateness) of the actual or measured intrinsic cardiac rate is used to switch from one state to another state according to the definition above. As VDD mode support in the at least one first state centers around the primary aim of supporting AV synchrony when relatively reliable intrinsic sensing input exists, the concept of up-to-dateness of the actual cardiac rate (also referred to as “freshness”) serves the needs of the VDD mode support outlined in this disclosure. A fresh/stale condition may be determined using weighted averaging of events and conditions using pre-defined criteria from a circular buffer of inputs or from an up/down counter that accumulates an average of weighted events/conditions. For example, it is observed whether the actual or measured intrinsic cardiac rate is determined from the present cardiac cycle or from previous cardiac cycles using an up/down counter which counts up if the actual cardiac rate stems from an atrial and/or ventricular intrinsic event of the present cycle or counts down if in the present cycle no intrinsic event was detected. Depending on the value of the counter, it is determined whether the calculated cardiac rate and/or the measured intrinsic or actual cardiac rate has to be regarded as fresh or stale.

The actual cardiac rate may be sourced by either intrinsic rhythm or by paced events. In our design, cardiac intervals are measured and separately intervals from cardiac cycles are measured which are only based on intrinsic timing and not paced events. This second group of measurements may be done from cycles where we do pace the ventricle if that pace was preceded by an atrial sense or by cycles in which there was a ventricular sense rather than a pace regardless of whether it was preceded by an atrial sense. So the actual cardiac rate may always be measured since there is always a ventricular event in every cardiac cycle and there is no need to determine whether our cardiac rate measurements are fresh or not. But the intrinsic cardiac rate is only measured on cycles which have intrinsic cardiac information and so this information may become stale.

Summary: Strategy is to distinguish between cardiac rates which are a property of the heart and cardiac rate measurements which are a property of the described system by using the word measurement when talking about the later, and to use the phrase intrinsic rate to mean rates which come from the sinus node in the heart and cardiac rate when talking about the actual heart rate whether it is controlled by the heart or the pacemaker.

In pacemaker embodiments where no direct measurement of atrial signaling is feasible (as, for example, when attempting to support VDD therapy using a leadless pacer wholly resident within the patient's right ventricle), the pacemaker may not always readily support atrial event detection with the same degree of reliability as traditional leaded systems. While the goal of the above pacemaker is to support VDD behavior whenever possible in the at least one first state, a state determining module of the processing unit is described that dynamically switches between at least one first state and several VDD mode sub-states (at least one second state, third state) to optimize the pacemaker timing behavior in accord with dynamically detected conditions associated with challenged access to reliable atrial input.

This is based on the observation that pacemakers must inherently always provide pacing support when intrinsic cardiac support is not detected. In the VDD mode, there is only ventricular pacing and potentially both atrial and ventricular sensing to detect the intrinsic activity. As long as no ventricular intrinsic events are detected, the device must periodically pace the ventricle. In the target patient for this device, there is typically always some intrinsic timing derived from the heart's sinus node. When the patient's AV conduction system fails, the sinus (atrial) timing and the pacemaker (ventricular) pace time can become un-synchronized. In this circumstance, the goal of the above pacemaker is to provide regular pacing support in the at least one first state subject to conditions where the atrial events are detectable and to provide pacing support in the at least one second state (while using timing that encourages the conditions in which intrinsic timing can be detected so that the heart's intrinsic activity and the pacing activity can be re-synchronized in the third state) subject to conditions where the atrial events are either wholly absent or undetectable. The third state is optimized for re-acquiring intrinsic rhythm information in cases where it has been absent, undetectable, or deliberately ignored for a duration due to suspected arrhythmias. When the intrinsic rhythm information has been acquired, the goal of the pacemaker is to time ventricular pacing such that the pace timing stays in sync with the sinus signal, which may be regular or may be intermittent. As the sinus rate changes, the pacemaker tracks/follows the sinus signaling so it can be followed, effectively keeping the ventricular pacing in sync.

Other states in this embodiment cover behaviors specific to the disengagement of atrial tracking if the atrial rate is suspected of being a tachyarrhythmia; replacement of intrinsic timing using a sensor rate correlated with metabolic demand; analysis sub-states intended to determine which timing conditions might provide more appropriate behaviors; and support for gradual rate transitions intended to assist in maximizing the potential for realizing improved patient comfort.

The pacemaker may be a conventional cardiac pacemaker or an ILP having the general structure as indicated above. Further, generally the units and components may work in time rates or may use corresponding time intervals. Accordingly, below explanations with regard to time intervals shall be understood to analogously refer to rates and vice versa.

With regard to the present invention, the processing unit is generally regarded as a functional unit of the pacemaker, that may interpret and/or execute instructions comprising an instruction control unit and an arithmetic and logic unit. The processing unit may comprise or be a microprocessor, a controller, a digital signal processor (DSP), an application specific integrated circuit (ASIC), a field-programmable gate array (FPGA), discrete logic circuitry or any combination thereof. Alternatively, or additionally, the processing unit may be realized using integrated dedicated hardware logic circuits, in particular, in the case of an ILP due to the small size and extreme power limitation. As indicated above, the processing unit comprises a state determining module, wherein the state determining module is configured to dynamically select one of at least one first state, at least one second state and a third state and to use the selected state to determine the ventricular pacing time and/or rate information to meet the patient's actual needs.

The processing unit processes signal data received from the first detector, for example, electrical signals of the patient's heart which are detected over time. In particular, the first detector is configured to detect the time-dependent electrical depolarization and repolarization field signals such as an electrocardiogram (ECG) or intracardiac electrogram (IEGM). These signals comprise signals caused by the depolarization of the atria (in the following, the intrinsic atrial event) and electrical signals caused by the depolarization of the ventricles (in the following, the intrinsic ventricular event). In the case of an ILP the intrinsic atrial event may be a far field electrical signal. The first detector may preprocess these data, for example, digitize the signals, filter them and/or amplify them.

The processing unit to which the electrical signals of the detector are transmitted perceives intrinsic ventricular events and intrinsic atrial events from the electrical signals, for example, the intrinsic atrial event from the P wave and the intrinsic ventricular event from the QRS complex. Since the far-field electrically measured P wave is much smaller in amplitude compared to near-field ventricle signals, a higher amplification may be used in a time period of the signal in which the P wave is expected than in the QRS and T-wave time intervals.

Alternatively, the electrical signal input provided by the first sensor may be split into two channels internal to the processing unit—one for the atrium and one for the ventricle—each channel having its own amplification and filtering schemes to respectively detect an intrinsic atrial event and an intrinsic ventricular event.

The pacemaker further comprises at least one second detector which is configured to detect time-dependent bodily signals of the patient different from the signals detected by the first detector, i.e., different from the time-dependent electric signals of the heart. The at least one second detector may detect atrial intrinsic events and/or ventricular intrinsic events and/or other signals, but not from electrical signals of the heart but from other sources. The second detector may be, for example, an accelerometer, a vibration sensor, an acoustic sensor (including ultrasound) and/or any other mechanical, electric and/or magnetic sensor that is capable of detecting time-dependent activity of the patient (e.g., motion sensor-based assessments of whether the patient is active, presenting a particular posture, realizing a specific activity level, or otherwise). The detector collects the activity signals of the patient and transforms them into electrical signals. Further, the detector may digitize analog signals, filter them and/or smooth them to reduce signal noise and/or cull specific metrics. Some pre-processing steps may be provided by the detector.

The signals received from the at least one second detector may be used by the state determining module to decide whether a switch from one state to another state is necessary according to the patient's present needs. This may be realized, for example, by a motion flag provided by the processing unit that is on if a patient activity is equal to and above a pre-defined level and off if a patient activity is below the pre-defined level.

The ILP or the conventional pacemaker is operated in the VDD pacing mode in the at least one first state. In the at least one first state, the pacemaker synchronizes ventricular pacing with the intrinsic atrial event based on fixed, pre-defined AV delay or an AV delay which is calculated on the basis of the actual cardiac rate. In an ILP that is implanted in the right ventricle, the atrial contraction information may be detected as a far field signal as indicated above.

The processing unit may further comprise a counter and a clock. The counter may be used to count cycles of the clock. The counter may be started at each sensed atrial or ventricular depolarization and count the number of clock cycles until the next atrial or ventricular depolarization occurs or ventricular pacing is provided by the pacing signal generator. In the at least one first state that uses VDD mode with atrial tracking, the processing unit determines the actual cardiac rate using the most recently detected ventricular event and is thereby continuously adapted to the patient's condition, wherein the actual or measured intrinsic cardiac rate is not changed with regard to the most recently determined value if, in one cycle, an intrinsic atrial event and/or an intrinsic ventricular event is not detected. For example, in the at least one first state, the processing unit may determine from the intrinsic atrial event and the intrinsic ventricular event or the ventricular pacing event the R-R interval of the actual cardiac cycle. Alternatively, a “current (average) R-R interval” may be determined as an average of a pre-defined number of previous R-R intervals of the previous cardiac cycles and of the R-R interval of the actual cardiac cycle, which may be weighted. In one embodiment, the R-R interval of the actual or measured intrinsic cardiac cycle is only used if at least one intrinsic event (an intrinsic atrial event and/or an intrinsic ventricular event) was used to determine the R-R interval of the respective cardiac cycle (i.e., a qualified R-R interval). The actual cardiac rate may be determined such that it is matched to the current (average) R-R interval or the R-R interval of the actual cardiac cycle.

In the third state the actual or measured intrinsic cardiac rate is determined analogously, if there is any intrinsic atrial event or any intrinsic ventricular event is detected for the respective cardiac cycle. If the actual or measured intrinsic cardiac rate is too stale, i.e., up-to-dateness of the actual cardiac rate is below a pre-defined threshold value, the actual or measured intrinsic cardiac rate is not used to determine the ventricular pacing time and/or rate information but a second pre-defined pacing rate.

As indicated above, in the at least one first state and in the third state, the ventricular pacing time and/or rate information may be determined from the actual or measured intrinsic cardiac rate. It is described that the actual cardiac rate and thereby the pacing time and/or rate information is/are continuously adapted to the patient's cardiac cycle condition thereby supporting synchronization of pacing and the heart's intrinsic activity, also for cycles where an intrinsic atrial event is not detected.

In the at least one second state the ventricular pacing time and/or rate information may be determined at least partly from signals of the at least one second detector and/or based on a pre-defined pacing rate, e.g., the resting rate. This includes the embodiment to use a pacing rate that is ramped from an initial (actual) cardiac rate to a pre-defined pacing rate, e.g., the resting rate or the motion sensor rate. The resting rate may be pre-defined for the specific patient, either directly or indirectly, by input from the HCP and stored in the data memory.

The “ventricular pacing time and/or rate information” includes any timing information the pacing signal generator needs to produce the ventricular pacing signal at the correct time, i.e., according to the treatment plan. The ventricular pacing time and/or rate information may be, in part, determined or dictated by an AV delay and/or a cardiac rate and/or the duration of a cardiac cycle. In one embodiment, the ventricular pacing time and/or rate information may comprise a hysteresis time to be added to the AV delay or the duration of a cardiac cycle, so that pacing is slightly delayed, to provoke a ventricular intrinsic event.

Based on the pacing control signal, the pacing signal generator produces the electrical pacing signal(s) to transfer it to the electrodes which apply the signal(s) to the heart's tissue adjacent to the electrode. The pacing signals are pulses that begin at a desired time point and have a desired intensity and duration. Further, the pulse waveform may be varied. Pacing control signaling is used by the processing unit to instruct the pacing signal generator on specifics associated with the duration, timing, and amplitude of the administered therapy output pulses. In particular, the ventricular pacing control signal contains ventricular pacing time and/or rate information. In one embodiment, the pacing signal is not determined (i.e., inhibited) or not transferred to the electrodes if an intrinsic ventricular event is detected within a predefined time period (i.e., the AV delay) after the detected intrinsic atrial event or an intrinsic ventricular event is detected within a predefined time period (i.e., the prevailing rate interval) subject to the lack of a detected atrial event in the at least one first state.

The pacemaker may comprise a data memory which may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The data memory saves the above and below mentioned thresholds and conditions. They are required by the processing unit during processing the above and below explained steps.

The pacemaker may comprise further modules such as a communication unit for communication with a remote computer and a power supply such as a battery. The communication unit may exchange messages with the external (at least partially extracorporeally) remote computer, for example, in one single direction or bidirectionally. The communication may be provided wirelessly via the patient's body, preferably acoustic, conducted and/or magnetically/inductively coupled, and/or the air using electromagnetic waves, for example, MICS-band, Bluetooth, WLAN, ZigBee, NFC, Wibree or WiMAX in the radio frequency region, or IrDA or free-space optical communication (FSO) in the infrared or optical frequency region or by wire (electrical and/or optical communication). The remote computer is a functional unit that can perform substantial computations, including numerous arithmetic operations and logic operations without human intervention, such as, for example, a personal mobile device (PMD), a desktop computer, a server computer, clusters/warehouse scale computer or embedded system. The pacemaker's units and components may be contained within a hermetically sealed housing.

In one embodiment the pacemaker comprises electrodes for application of an electrical pacing signal provided by the pacing signal generator. The electrodes are electrically connected to the pacing signal generator via a header of the pacemaker. In one embodiment (i.e., in the case in which the pacemaker is a conventional pacemaker) the electrode may comprise a lead which may be detachably connected to the respective connector at the header. With regard to an ILP one electrode may be located at a distal end of the ILP, close to a fixation member by which the ILP is fixed in the tissue of the patient's heart, for example, against or within the tissue of a ventricle. A second electrode may be located at the proximal end of the ILP or a part of the ILP housing that may, for example, serve as counter electrode. Further, the electrodes may be adapted to detect the intrinsic ventricular event or the intrinsic atrial event in each case over time by picking up electrical potentials. The electrodes may thereby be part of the detector of the pacemaker.

In the at least one first state, the pacing mode supported by the pacemaker is, as explained above, VDD with atrial tracking. This mode assumes that the patient has some form of intrinsic AV conduction disorder, either complete or intermittent. It also assumes that the sinus node is generally competent, and the VDD mode attempts to track the sinus rate and to provide AV synchronization. The AV delay may be determined based on the actual cardiac rate and may be set to the usual time difference between the intrinsic atrial and ventricular contraction or depolarization. From the actual cardiac rate, the current AV delay may be determined by a known calculation or using a look-up-table contained in the data memory. The current AV delay may change with the actual cardiac rate depending on whether or not a dynamic or fixed, pre-defined AV delay is used.

The pacemaker according to the above embodiment supports VDD behavior in the first state when possible and dynamically switches between several sub-states (at least one second state, third state) to optimize the pacemaker timing behavior in accord with dynamically detected conditions. These states are controlled by the state determining module (also referred to as state machine in the following).

In one embodiment, the state determining module is configured such that at least one of the second states is an intermediate state used (e.g., directly) after leaving the at least one first state, for example, when an SVT is suspected, wherein in the intermediate state the ventricular pacing time and/or rate information is determined from ramping an initial cardiac rate to the first pre-defined pacing rate, e.g., the resting rate of the patient. The initial cardiac rate may be the actual cardiac rate of the time point at which the state determining module switched from the at least one first state to the respective second state. The intermediate state is left to another second state as soon as the first pre-defined pacing rate is reached. Such state switch transitions aim to ensure the administration of baseline bradycardia mitigation therapy while also striving to adapt to measurable patient needs; minimize the introduction of noticeable symptoms thought the avoidance of abrupt rate changes; and return device/patient interactions to optimal conditions with minimal delay.

In one embodiment, the state determining module is configured such that it selects one of the at least one second state that uses the signals received from the at least one second detector for the determination of the ventricular pacing time and/or rate information depending on whether the usage of the respective second detector is allowed, for example, by the HCP. This embodiment has the advantage that the HCP may adapt the operation of the cardiac pacemaker better to the patient's needs. Additionally, if motion-dependent pacing is not needed for the respective patient, the pacemaker may avoid directing power from its limited-capacity primary cell chemistry to run the accelerometer. Furthermore and generally, in certain second states where patient motion is tracked, the system is structured to turn off any capacity to monitor atrial input signaling. Thereby the longevity of the power source (battery) is increased.

In one embodiment, the state determining module is configured to compare a pre-defined cardiac check rate with the actual cardiac rate and/or a pre-defined freshness value with a value representing the up-to-dateness of the actual cardiac rate and/or a timeout duration with a time period elapsed since a pre-defined time point and to assess based on at least one of these comparisons or signaling from a momentarily activated second detector whether the actual state still meets the patient's needs or whether the actual state needs to be left.

At least the above problem is further solved by an operation method of a cardiac pacemaker for a patient's heart comprising a processing unit with a data memory, further comprising a first detector, and at least one second detector, a pacing signal generator, which are all electrically connected to the processing unit, wherein time-dependent electrical signals of the heart, for example, an intra-cardiac electrogram, are detected by the first detector, wherein time-dependent bodily signals of the patient different from the signals detected by the first detector are detected by the at least one second detector, wherein the detected and, if applicable, pre-processed signals are transmitted to the processing unit by the first detector and by the at least one second detector, wherein the processing unit processes the signals received from the first detector and the signals from the at least one second detector and detects from the received signals of the first detector intrinsic atrial events, intrinsic ventricular events and pacing events and determines an actual or measured intrinsic cardiac rate, wherein the processing unit comprises a state determining module, wherein one of at least one first state, at least one second state and a third state is dynamically selected by the state determining module and the selected state is used to determine the ventricular pacing time and/or rate information to meet the patient's actual needs,
wherein in the at least one first state the actual cardiac rate is determined from the detected atrial intrinsic events, ventricular intrinsic events and/or pacing events and the current ventricular pacing time and/or rate information is determined in a VDD mode with atrial tracking based on the determined actual cardiac rate,
wherein in the at least one second state the determination of the actual cardiac rate from the (detected) atrial intrinsic events, ventricular intrinsic events and/or, if applicable, in one embodiment also from pacing events is prohibited and the ventricular pacing time and/or rate information is determined based at least partly on the signals received from the at least one second detector and/or based on a first pre-defined pacing rate,
wherein in the third state the actual cardiac rate is determined from the detected atrial intrinsic events, ventricular intrinsic events and pacing events and the ventricular pacing time and/or rate information is determined based on a second pre-defined pacing rate or the determined actual cardiac rate dependent on an up-to-dateness of the actual cardiac rate, wherein the state determining module switches from one state to another state of said states, wherein switching from the at least one first state directly to the third state is prohibited, depending on the present state and on at least one of a set of pre-defined conditions.

In one embodiment of the method, the processing unit processes the signals received from the first detector and the signals from the at least one second detector and detects from the received signals of the first detector intrinsic atrial events, intrinsic ventricular events and pacing events and determines the actual cardiac rate together with an information on an up-to-dateness of the actual cardiac rate, wherein the up-to-dateness determination is based on the detected intrinsic atrial events and intrinsic ventricular events, wherein the processing unit determines and transmits a ventricular pacing time and/or rate information to the pacing signal generator for providing a pacing signal for the patient's heart based on this information.

As said above, the actual cardiac rate may also be called as measured intrinsic cardiac rate and vice versa.

In one embodiment of the method, the set of pre-defined conditions comprises at least two of the following conditions:
the up-to-dateness of the measured intrinsic or actual cardiac rate,
a comparison of the actual cardiac rate and a pre-defined upper rate threshold or a pre-defined lower rate threshold,
the signals received from the at least one second detector, and
a comparison of a time elapsed from a pre-defined time point in connection with the switch to the present state and a pre-defined time interval threshold.

In one embodiment of the method, at least one of the second states is an intermediate state that is used by the state determining module (e.g., directly) after leaving the at least one first state, wherein in the intermediate state the ventricular pacing time and/or rate information is determined from ramping an initial cardiac rate to the first pre-defined pacing rate.

In one embodiment of the method, one of the at least one second state that uses the signals received from the at least one second detector is selected by the state determining module for the determination of the ventricular pacing time and/or rate information depending on whether the usage of the respective second detector is allowed, for example, by the HCP.

In one embodiment of the method, in the at least one second state the ventricular pacing time and/or rate information is determined using a (e.g., nominal) VVI behavior or a VVI-R behavior by the state determining module.

As indicated above, in one embodiment in the VDD mode with atrial tracking used in the at least one first state to determine the ventricular pacing time and/or rate information the actual cardiac rate is determined using the most recently detected at least one intrinsic atrial event and the most recently detected at least one intrinsic ventricular event and is thereby continuously adapted to the patient's condition, wherein the actual cardiac rate is not changed with regard to the most recently determined value if, in one cycle, an intrinsic atrial event and/or an intrinsic ventricular event is not detected.

The above embodiments of the operation method have the same advantages as the above pacemaker. Embodiments of the pacemaker indicated above may be realized in the operation method analogously. It is referred to the above explanation of the cardiac pacemaker in this regard.

The above method is, for example, realized as a computer program (i.e., represents computer program code) which comprises instructions which, when executed, cause the processing unit (processor) to perform the steps of the above method (to be executed by the cardiac pacemaker, in particular at its processing unit) which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by the processing unit, cause the processing unit to perform the steps of the above defined method. Accordingly, a computer readable data carrier storing such computer program product is disclosed.

The above cardiac pacemaker, method, computer program and computer program product provide an approach to VDD mode support (in the at least one first state) that offers a switching between a large collection of sub-modes or sub-states within the VDD mode (which demonstrate behaviors similar to different modes but which are all part of the broad umbrella of a VDD mode setting), wherein the extent of the sub-modes included stems from user configuration settings. Thereby, the cardiac pacemaker can leverage clinician configuration settings as well as the inputs from the pacemaker's surrounding environment to compartmentalize the use of different power-consuming system design attributes and cater therapy support to patient needs.

In addition to state machine control of various sub-modes, the cardiac pacemaker embodies combining of signals from various status sources into a dynamic selector for the timing pattern most suited for the current conditions. One aspect is the history-based degree to which collected intrinsic cardiac timing information is relevant which is derived from atrial and ventricular senses. Intrinsic senses in successive cardiac intervals provides intrinsic rate information and individual senses provide phasing information (i.e., the offset within the cardiac cycle between the atrial event and the ventricular event). Certain cardiac rhythms represent irregular timing which decrease the confidence that the calculated timing is accurate. These conditions are distilled with logic into indications of the (average) actual cardiac rate, phasing and freshness. The at least one second sensor which measures external activities (e.g., accelerometer) also provides information which can increase or decrease the confidence in the cardiac status, if the HCP or the detailed algorithm deems it suitable for the specific patient. These signals are processed and mapped into triggers that control state transitions and the calculated rhythms are used to smooth over missing intrinsic timing information.

The core conceptual suite of interactions handled by the VDD mode support used in the pacemaker, as detailed in the list below, operates as a balancing act between a multitude of therapy optimization needs.

1.) If atrial senses are sufficiently reliable and recent (i.e., “fresh”), the VDD mode support ultimately enters and holds in a synchronous tracking condition in the at least one first state.

2.) If atrial senses are occasionally missing, the VDD mode support persists the recent rate history to prop the therapy output and hold within a synchronous tracking condition in the at least one first state.

3.) When in a tracking condition in the at least one first state, the VDD mode support monitors for signatures suggestive of SVTs and either continues to track (lacking suspicion) or migrates toward low-rate idled conditions (through interim states, when suspected) to await SVT termination in the at least one second state.

4.) If atrial senses are unreliable and the clinician wishes to utilize motion inputs, the VDD mode support is configured to permit the therapy rate to be driven by accelerometer input signaling in the at least one second state.

5.) If atrial senses have been unreliable and the clinician wishes to avoid using motion inputs or the patient has simply been inactive, the VDD mode support may idle at a low rate in the at least one second state and attempts to resynchronize in the third state.

6.) The state determining module manages any and all transitions between the multitude of stable sub-states within in a manner intended to mitigate large sudden rate changes that would be symptomatic to the patient by using intermediate states described above.

7.) The state determining module additionally intentionally disrupts the sub-state residence in a periodic fashion through state transitions that aim to ensure that the therapy avoids remaining unwittingly “stuck” in non-ideal conditions.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein

FIG. 1 shows an embodiment of a cardiac pacemaker within a cross section of a patient's heart,

FIG. 2 depicts a functional block diagram of the pacemaker shown in FIG. 1,

FIG. 3 shows an enlarged side view of the pacemaker of FIG. 1, and

FIG. 4 shows a flow chart of an embodiment of the operation method of the state determining module of the pacemaker of FIG. 1.

### DETAILED DESCRIPTION

In the following, the present invention is described with regard to an ILP. It may analogously be realized in a conventional pacemaker, as well.

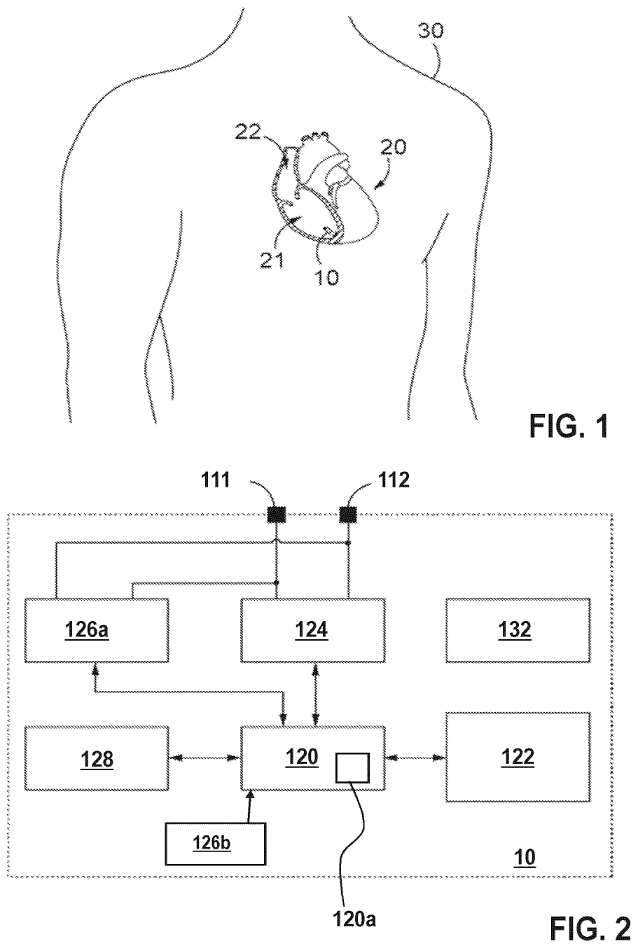

FIG. 1 shows an example leadless ventricular pacemaker (ILP) 10 implanted within the heart 20 of a patient 30. ILP 10 may be configured to be implanted within the right ventricle 21 of the heart 20 and pace this ventricle, sense intrinsic ventricular depolarizations and depolarizations of the atria (e.g., the right atrium 22) and inhibit ventricular pacing in response to detected ventricular depolarization. A programmer (not shown) may be used to program ILP 10 and retrieve data from ILP 10 via a wireless communication connection for which examples are explained above. The ILP 10 is one example of a cardiac pacemaker 10. Other embodiments of the cardiac pacemaker 10 are possible.

FIG. 2 shows a functional block diagram of the ILP 10 configured for implantation within right ventricle 21 (FIG. 1). The ILP 10 comprises a processing unit 120 with a clock, at least one counter for the clock signals and a data memory 122, a pacing signal generator 124, a detector unit comprising a first detector 126a and at least one second detector 126b, a communication unit 128, and a power source 132. The power source 132 may be electrically connected to one or more of the other components 120, 122, 124, 126a, 126b, 128 (not shown in FIG. 2) and may include a battery, e.g., a rechargeable or non-rechargeable battery. The power source provides electrical energy to all units and components of the ILP 10 (not explicitly shown in the figure), in particular to all units mentioned above and is therefore electrically connected to these units and components. Similar or identical units and functionality may also be included in the ILP 10. Units of the pacemaker of present disclosure may include any discrete and/or integrated electronic circuit components that implement analog and/or digital circuits capable of producing the functions attributed to the units herein. For example, the units may include analog circuits, e.g., amplification circuits, filtering circuits, and/or other signal conditioning circuits. The units may also include digital circuits, e.g., combinational or sequential logic circuits, memory devices, etc. The units may further be realized using integrated dedicated hardware logic circuits. The data memory 122 may include any volatile, non-volatile, magnetic, or electrical media mentioned above.

Furthermore, the processing unit 120 may include instructions that, when executed by one or more processing circuits, cause the units to perform various functions attributed to these units herein. The functions attributed to the units herein may be embodied as one or more processors, hardware, firmware, software, or any combination thereof. Depiction of different features as units is intended to highlight different functional aspects and does not necessarily imply that such units must be realized by separate hardware or software components. Rather, functionality associated with one or more units may be performed by separate hardware or software components or integrated within common or separate hardware or software components. Data memory 122 may store computer-readable instructions that, when executed by processing unit 120, cause processing unit 120 to perform the various functions attributed to processing unit 120 herein. Further, data memory 122 may store parameters for these functions, e.g., pacing signal parameters, conditions and thresholds described above and below. The pacing instructions and pacing signal parameters, conditions and thresholds may be updated by the programmer using the communication unit 128. The communication unit 128 may comprise an antenna, coil, patient anatomical interface, and/or a transceiver.

The processing unit 120 may communicate with pacing signal generator 124 and detectors 126a,b thereby transmitting signals. Pacing signal generator 124 and the first detector 126a are electrically coupled to electrodes 111, 112 of the ILP 10. The first detector 126a is configured to monitor signals from electrodes 111, 112 to monitor electrical activity of heart 20. The second detector 126b may be realized as a motion sensor, for example, an accelerometer or any other motion sensor described above. However, an accelerometer-based motion sensor does not necessarily require any connection with the electrodes 111, 112. The motion sensor collects time-dependent motion signals as known from an accelerometer and transmits these signals to the processing unit 120, wherein the signals may be pre-processed similarly to the signals of the first detector 126a as described below. Pacing signal generator 124 is configured to deliver electrical stimulation signals to ventricle 21 via electrodes 111, 112. Processing unit 120 may control pacing signal generator 124 to generate and deliver electrical stimulation to ventricle 21 via electrodes 111, 112. Electrical stimulation may include pacing pulses. Processing unit 120 may control pacing signal generator 124 to deliver electrical stimulation therapy according to one or more therapy programs including pacing parameters, which may be stored in data memory 122.

The first detector 126a may further include circuits that acquire time-dependent electrical signals (e.g., electric depolarization and repolarization signals) from the heart including intrinsic cardiac electrical activity, such as intrinsic atrial event and, if applicable, an intrinsic ventricular event. The first detector 126a may filter, amplify, and digitize the acquired electrical signals of the heart chambers contractions and include support for splitting inputs from the electrodes into multiple channels for subsequent handling by 120. Processing unit 120 may receive the intrinsic atrial event and, if applicable, the intrinsic ventricular event generated by first detector 126a.

Processing unit 120 may assess the intrinsic atrial event and, if applicable, the intrinsic ventricular event received from the first detector 126a and is configured to determine the (time-dependent) actual cardiac rate.

ILP 10 may include a housing, anchoring fixation features (tines) 107, and the electrodes 111, 112 at a first end 10a and near a second end 10b of the ILP 10. The housing may have a pill-shaped cylindrical form factor in some examples. The anchoring fixation features 107 are configured to connect ILP 10 to heart 20. Anchoring fixation features 107 may be fabricated from a shape memory material, such as Nitinol. In some examples, anchoring fixation features may connect ILP 10 to heart 20 within one of the chambers of heart 20. For example, as illustrated and described herein with respect to FIG. 1, anchoring fixation features 107 may be configured to anchor ILP 10 to heart 20 within right ventricle 21. Although ILP 10 includes a plurality of anchoring fixation features/elements 107 that are configured to stably engage ILP 10 to cardiac tissue in the right ventricle, it is contemplated that a pacemaker according to the present disclosure may be engaged with cardiac tissue in other chambers of a patient's heart 20 using other types of anchoring fixation features. The housing further comprises a catheter engagement hitch 115 at the second end 10b of the ILP 10.

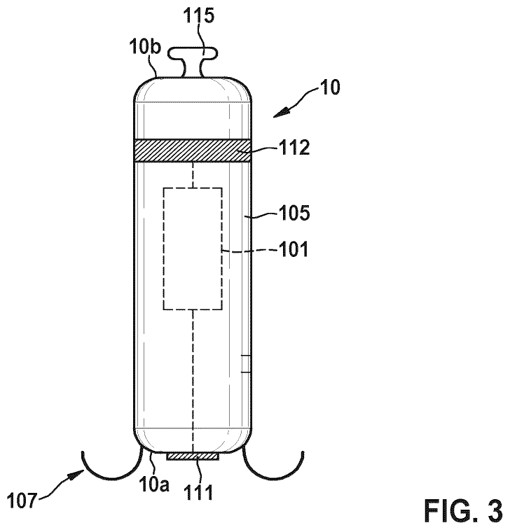

ILP 10 may include two electrodes 111, 112, although more than two electrodes may be included on a pacemaker in other examples. As depicted in FIG. 3 electrodes 111, 112 may be spaced apart a sufficient distance to be able to detect various electrical signals generated by the heart 20, such as P-waves generated by atria and QRS complex generated by ventricles. The housing houses electronic components of ILP 10. Electronic components may include any discrete and/or integrated electronic circuit components that implement analog and/or digital circuits capable of producing the functions attributed to ILP 10 described above.

The communication unit 128 may enable ILP 10 to communicate with other electronic devices, such as a programmer or other external patient monitor. In some examples, the housing may house a coil and/or an antenna for wireless communication. Housing may also include the power source 132.

The processing unit 120 may be adapted to control pacing of the right ventricle 21 in the first state using the known VDD mode based on the intrinsic atrial event containing atrial contractions and, if applicable, the intrinsic ventricular event containing ventricular contractions. The counter of the processing unit 120 used to time the AV delay (for providing the ventricular pace signal) may also be used to measure intrinsic AV delays. The VDD pacing mode may be R-Sync in the ILP 10. This means that every cycle is synchronized by every used ventricular event (intrinsic ventricular contraction or ventricular pacing). It is also an atrial tracking mode. This means that every sensed atrial contraction can shift the timing. In other words, VDD is both R-Sync and P-Sync. The timing of the next potential ventricular pacing signal is scheduled based on the most recent ventricular event and a targeted pacing interval (determined from a target cardiac rate). Sensed atrial contractions “re-schedule” the next pacing signal by starting an AV interval. The actual cardiac rate is determined from the intrinsic atrial event and, if applicable, from the intrinsic ventricular event and/or from the paced output from the pacemaker (ILP 10). To control the pacing, the processing unit comprises a state determining module 120a (see FIG. 2) which represents a part of the algorithm that is used to dynamically switch between a first state representing the VDD mode and several second states (in the following, sub-states) to determine the ventricular pacing time and/or rate information dependent on the present condition of the patient and key parameters pre-defined by the HCP.

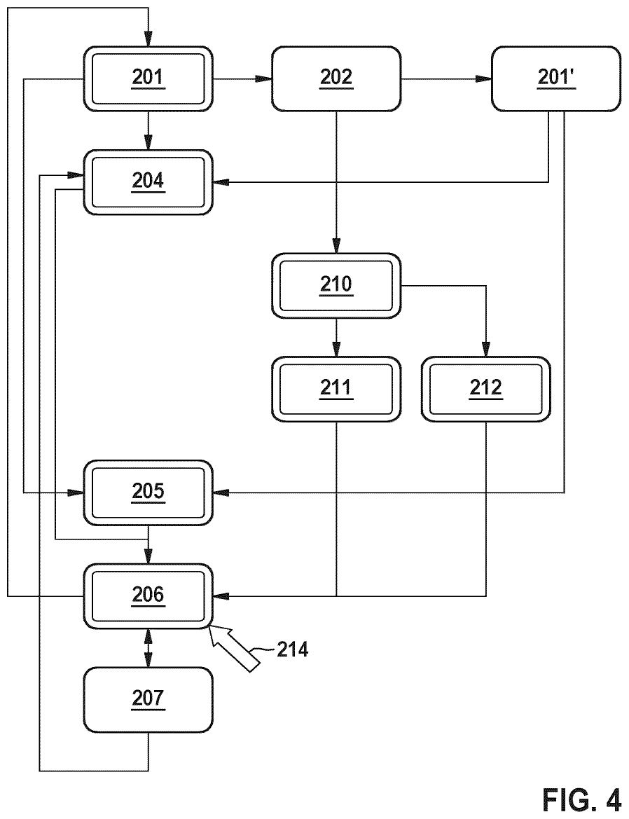

The method shown in diagram of FIG. 4 depicts an example of the operation of such state determining module controlling the pacing interval source, further referring to the tracking interval behavior, the motion circuit (enabled/disabled), the atrial tracking behavior (atrial sensing enabled/disabled), and the rate fading behavior under the various conditions that may arise while the pacemaker is programmed to the VDD mode. The ideal behavior in the VDD mode represented by a first state 201 of the state determining module of the processing unit is to have the ventricular event (either an intrinsic ventricular contraction or a ventricular pacing signal if the intrinsic AV conduction is inadequate) track the intrinsic atrial contractions. If an intrinsic ventricular contraction occurs before the AV timeout provided dependent on the actual cardiac rate, the pacemaker stops the AV delay and waits for the next atrial contraction in the atrial intrinsic event. If the AV timeout occurs, a ventricular pacing signal is delivered. The (unchecked) atrial tracking is symbolized by the first state 201 in FIG. 4 and uses the signals provided by the first detector 126a. When the atrial rate and thereby the actual cardiac rate starts ramping up, the timing system tracks it under the assumption that it represents an increased cardiac demand due to the activity of the patient. It is also possible that the actual cardiac rate is the result of an SVT, in which case it may be undesirable to track it. That's why the state determining module switches to check at 202 if the actual cardiac rate is greater than or equal to a pre-defined validity check rate, which may be, for example, 85 bpm. As long as the actual cardiac rate is less than the validity check rate, VDD mode pacing in the first state is continued (state 201). If the actual cardiac rate is equal to or greater than the validity check rate it is further checked in check 202 whether a motion flag is on or off, wherein the motion flag is on if activity is detected by the second detector 126b equal to or above a pre-defined level, whereas the motion flag is off if activity is a below a pre-defined level. The state of the motion flag is determined by the processing unit 120 using the motion signal as indicated above from the second detector 126b (e.g., an accelerometer). The motion sensor provides an alternate indication of an increased cardiac demand.

If the motion flag indicates activity (i.e., the motion flag is on), then the cardiac rate is considered to be a valid demand rate and the tracking behavior continues. The procedure continues with the first state 201′, a VDD mode and atrial tracking. This state is now regarded as checked with regard to motion of the patient to avoid SVT tracking (see step 201′ in FIG. 4). After a pre-defined time interval and/or if the actual cardiac rate is less than the resting rate, the first state 201′ may be left and the state determining module returns to the first state 201.

In the first (checked) state 201′ and in the (initial) first state 201, both with atrial tracking, the determined actual cardiac rate may further be subject to an upper tracking rate which is a patient specific rate as a maximum cardiac rate not to exceed, i.e., the paced rate is limited to the programmed upper tracking rate.

In the first state 201, 201′, in particular if a pre-defined maximum rate is used, it is continuously checked whether the ventricular paces are in synchronization with the atrial contractions. This is advantageous as the heart's activity may lead to asynchronized behavior which will result in missed atrial senses (indicated by “loss of freshness/stale”). The actual or measured intrinsic cardiac rate is subject to a continuing freshness check, i.e., whether the actual or measured intrinsic cardiac rate is fresh or stale as explained below.

In the atrial tracking state, continuously measured qualified R-R intervals are used to determine the measured intrinsic cardiac rate as indicated above. The measured intrinsic cardiac rate is regarded stale if it is based on detected intrinsic atrial and/or ventricular events from previous cycles that dates back a pre-defined number of cycles. The processing unit 120 may provide a counter to count the number of cycles without intrinsic atrial and/or ventricular events and thereby detects whether the actual measured intrinsic cardiac rate is fresh or stale.

In case a stale intrinsic cardiac rate is detected by the processing unit 120 the state determining module will switch the first state 201, 201′ (atrial tracking state, checked or unchecked) to either a sensor state 204, if the motion sensor is enabled as a backup rate control by the HCP, or to a fade to rest state 205 if the motion sensor is not enabled as a backup rate control. In the fade to rest state 205 the cardiac rate is step-by-step decreased (ramped down) to a pre-defined resting rate. Atrial senses are ignored in this state. The sensor state 204 and the fade to rest state 205 represent a second state according to the definition in the general part of the description.

If the sensor state 204 is active, atrial senses are ignored, as well. State 204 effectively is based on a VVI-R behavior. When the motion sensor rate drops down to the resting rate, the state determining module 120a switches back to a third state (FindSync state 206) and begins monitoring for intrinsic atrial activity again. While in the FindSync state 206, lack of both atrial sensing and AV conduction or atrial arrhythmias may prevent locking back into the atrial tracking first state 201. This may be realized, for example, by using the resting rate as the pacing rate. Additionally, the first detector 126a is used to detect intrinsic atrial senses. If an atrial sense is detected, an AV delay is started to phase-shift the pacemaker activity to align it with the cardiac activity. If at least two consecutive cycles are found in which either atrial or ventricular intrinsic senses are detected, the intrinsic VV interval and thereby the intrinsic cardiac rate can be measured and used to set the ventricular pacing time and/or rate information. If this proves reliable and is checked by some pre-defined conditions with regard to plausibility, the state determining module 120a switches to the first state 201 (atrial tracking, unchecked). The timing system will occasionally turn on the motion sensor while in the 206 state (temporarily switching to the patient activity check (step 207)) to detect whether there is a cardiac demand condition that is not being satisfied by the FindSync behavior. If this is detected, the state determining module 120a will switch to the sensor state 204 until the motion sensor rate again drops back to the resting state.

If the check in step 202 determines that the increasing atrial rate might be an SVT, which it does by finding that the motion flag is off even though the cardiac rate is greater than or equal to the validity check rate, the state determining module 120a is directed to second, non-tracking states to avoid tracking the arrhythmia. The state following the check 202 is the SVT state or Fade-to-Non-tracking state 210 (second state). In this state the cardiac rate is ramped down from the actual cardiac rate to the resting rate. If the resting rate is reached and the HCP has decided not to use motion inputs for mode switched conditions, the state determining module 120a continues with the non-tracking state 211 which provides VVI behavior (non-tracking state 211 is another second state). In the states 210 and 211 atrial detections by the first sensor 126a are disabled, and the second sensor 126b is also disabled according to a preselection of a respective option the HCP. After a predefined or user-selectable hold-off time period is expired while the non-tracking state 211 is adopted the processing unit enters the FindSync state 206 (third state). The hold-off time period begins or initiates at either the Tracking Validity Check failure or once the lower rate is reached. The durations of this period may be programmable and/or between 10 and 20 minutes, preferably 15 minutes. From the FindSync state 206 the processing unit may continue with the first state 201 if synchronization with the intrinsic atrial event was successful.

If the HCP has selected the option where the motion sensor can be used as a secondary rate response source, another second state, the non-tracking with motion state 212 is selected, providing VVIR behavior, after the resting rate (RR) was reached in the Fade-to-Non-Tracking state 210. In the non-tracking with motion state, 212, the atrial sensing by the first detector 126a is disabled, and the second detector 126b (motion sensor) is enabled. When the motion sensor is being used, the maximum pacing rate is the programmed maximal sensor rate (MSR).

The next state for the non-tracking state 211 is the Find Sync state 206 (third state), which, as noted above, is only entered after a timeout expiration. If the system resides in the non-tracking with motion state, 212, instead, after a timeout expiration, the sensor state 204 is entered. After entering the sensor state, 204, once the sensor-driven pacing rate matches the resting rate, the FindSync state 206 is entered. From the FindSync state 206 the state determining module 120a may continue with the first state 201 (atrial tracking, unchecked) as indicated above.

The entry point for the pacemaker entering the state determining module after initializing the system and start of the pacing is the FindSync state 206. This is symbolized in FIG. 3 by arrow 214.

The above explained embodiment of a cardiac pacemaker (ILP 10) and respective operation method realizes a means to leverage “at-a-distance” far-field IEGM input (which is known to be sub-optimally reliable compared to direct leaded measurements) to facilitate a VDD mode support capability within a stand-alone leadless pacemaker that largely mimics comparable therapeutic offerings in traditional leaded pocket-based IPGs-effectively provisioning dual-chamber therapy from a device wholly resident within a single heart chamber. Additionally, the outlined VDD mode support architecture offers a means for patients with AV block to benefit from a device type whose implantation proves less invasive than legacy formats and places smaller therapeutic device volumes within patient physiology—extending state of the art CRM offerings to patient sub-populations beyond those simply suffering from bradycardia paired with chronic AF. Furthermore, the cardiac pacemaker and the operation method prove feasible to support atrial tracking at higher rates due to the employment of electrical means affiliated with myocardial depolarization as opposed to the subsequent mechanical signatures affiliated with atrial contraction responses.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A cardiac pacemaker for a patient's heart comprising a processing unit with a data memory, further comprising a first detector, and at least one second detector, a pacing signal generator, which are all electrically connected to the processing unit, wherein the first detector is configured to detect time-dependent electrical signals of the heart, wherein the at least one second detector is configured to detect time-dependent bodily signals of the patient different from the signals detected by the first detector, wherein the first detector and the at least one second detector are configured to transmit the detected and, if applicable, pre-processed signals to the processing unit, wherein the processing unit is configured to process the signals received from the first detector and the signals from the at least one second detector and to detect from the received signals of the first detector intrinsic atrial events, intrinsic ventricular events and pacing events and to determine an actual cardiac rate, wherein the processing unit comprises a state determining module, wherein the state determining module is configured to dynamically select one of at least one first state at least one second state and a third state and to use the selected state to determine the ventricular pacing time and/or rate information to meet the patient's actual needs,
wherein in the at least one first state the actual cardiac rate is determined from the detected atrial intrinsic events, and/or ventricular intrinsic events and the current ventricular pacing time and/or rate information is determined in a VDD mode with atrial tracking based on the determined actual cardiac rate,
wherein in the at least one second state the determination of the actual cardiac rate from the intrinsic atrial and ventricular events is prohibited and the ventricular pacing time and/or rate information is determined based at least partly on the signals received from the at least one second detector and/or based on a first pre-defined pacing rate,
wherein in the third state the actual cardiac rate is determined from the detected intrinsic atrial events, intrinsic ventricular events and pacing events and the ventricular pacing time and/or rate information is determined based on a second pre-defined pacing rate or the determined actual cardiac rate dependent on the up-to-dateness of the actual cardiac rate,
wherein the state determining module is configured to switch from one state to another state of said states, wherein switching from the at least one first state directly to the third state is prohibited, depending on the present state and on at least one of a set of pre-defined conditions.

2. The cardiac pacemaker of claim 1, wherein the processing unit is configured to process the signals received from the first detector and the signals from the at least one second detector and to detect from the received signals of the first detector intrinsic atrial events, intrinsic ventricular events and pacing events and to determine the actual cardiac rate together with an information on an up-to-dateness of the actual cardiac rate, wherein the up-to-dateness determination is based on the detected intrinsic atrial events and intrinsic ventricular events, wherein the processing unit is configured to determine and transmit a ventricular pacing time and/or rate information to the pacing signal generator for providing a pacing signal for the patient's heart based on this information.

3. The cardiac pacemaker of claim 1, wherein
the set of pre-defined conditions comprises at least two of the following conditions:
the up-to-dateness of the actual cardiac rate,
a comparison of the actual cardiac rate and a pre-defined upper rate threshold or a pre-defined lower rate threshold,
the signals received from the at least one second detector, and
a comparison of a time elapsed from a pre-defined time point in connection with the switch to the present state and a pre-defined time interval threshold.

4. The cardiac pacemaker of claim 1, wherein the state determining module is configured such that at least one of the second states is an intermediate state used after leaving the at least one first state, wherein in the intermediate state the ventricular pacing time and/or rate information is determined from ramping an initial cardiac rate to the first pre-defined pacing rate.

5. The cardiac pacemaker of claim 1, wherein the state determining module is configured such that it selects one of the at least one second state that uses the signals received from the at least one second detector for the determination of the ventricular pacing time and/or rate information depending on whether the usage of the respective second detector is allowed.

6. The cardiac pacemaker of claim 1, wherein the state determining module is configured such that in the at least one second state the ventricular pacing time and/or rate information is determined using a VVI-behavior or a VVI-R behavior.

7. An operation method of a cardiac pacemaker for a patient's heart comprising a processing unit with a data memory, further comprising a first detector, and at least one second detector, a pacing signal generator, which are all electrically connected to the processing unit, wherein time-dependent electrical signals of the heart, are detected by the first detector, wherein time-dependent bodily signals of the patient different from the signals detected by the first detector are detected by the at least one second detector, wherein the detected and, if applicable, pre-processed signals are transmitted to the processing unit by the first detector and by the at least one second detector, wherein the processing unit processes the signals received from the first detector and the signals from the at least one second detector and detects from the received signals of the first detector intrinsic atrial events, intrinsic ventricular events and pacing events and determines an actual cardiac rate, wherein the processing unit comprises a state determining module, wherein one of at least one first state, at least one second state and a third state is dynamically selected by the state determining module and the selected state is used to determine the ventricular pacing time and/or rate information to meet the patient's actual needs,
wherein in the at least one first state the actual cardiac rate is determined from the detected atrial intrinsic events and ventricular intrinsic events and the current ventricular pacing time and/or rate information is determined in a VDD mode with atrial tracking based on the determined actual cardiac rate,
wherein in the at least one second state the determination the actual cardiac rate from the intrinsic atrial events and intrinsic ventricular events is prohibited and the ventricular pacing time and/or rate information is determined based at least partly on the signals received from the at least one second detector and/or based on a first pre-defined pacing rate,
wherein in the third state the actual cardiac rate is determined from the detected intrinsic atrial events, intrinsic ventricular events and pacing events and the ventricular pacing time and/or rate information is determined based on a second pre-defined pacing rate or the actual cardiac rate dependent on an up-to-dateness of the actual cardiac rate,
wherein the state determining module switches from one state to another state of said states, wherein switching from the at least one first state directly to the third state is prohibited, depending on the present state and on at least one of a set of pre-defined conditions.

8. The method of claim 7, wherein the processing unit processes the signals received from the first detector and the signals from the at least one second detector and detects from the received signals of the first detector intrinsic atrial events, intrinsic ventricular events and pacing events and determines the actual cardiac rate together with an information on an up-to-dateness of the actual cardiac rate, wherein the up-to-dateness determination is based on the detected intrinsic atrial events and intrinsic ventricular events, wherein the processing unit determines and transmits a ventricular pacing time and/or rate information to the pacing signal generator for providing a pacing signal for the patient's heart based on this information.

9. The method of claim 7, wherein the set of pre-defined conditions comprises at least two of the following conditions:
the up-to-dateness of the actual cardiac rate,
a comparison of the actual cardiac rate and a pre-defined upper rate threshold or a pre-defined lower rate threshold,
the signals received from the at least one second detector, and
a comparison of a time elapsed from a pre-defined time point in connection with the switch to the present state and a pre-defined time interval threshold.

10. The method of claim 7, wherein at least one of the second states is an intermediate state that is used by the state determining module after leaving the at least one first state, wherein in the intermediate state the ventricular pacing time and/or rate information is determined from ramping an initial cardiac rate to the first pre-defined pacing rate.

11. The method of claim 7, wherein one of the at least one second state that uses the signals received from the at least one second detector is selected by the state determining module for the determination of the ventricular pacing time and/or rate information depending on whether the usage of the respective second detector is allowed.

12. The method of claim 7, wherein in the at least one second state the ventricular pacing time and/or rate information is determined using a VVI-behavior or a VVI-R behavior by the state determining module.

13. The method of claim 7, wherein in the VDD mode with atrial tracking used in the at least one first state to determine the ventricular pacing time and/or rate information the actual cardiac rate is determined using the most recently detected atrial event and/or the most recently detected intrinsic ventricular event and is thereby continuously adapted to the patient's condition, wherein the actual cardiac rate is not changed with regard to the most recently determined value if, in one cycle, an intrinsic atrial event and/or an intrinsic ventricular event is not detected.

14. A computer program product comprising instructions which, when executed by a processing unit, cause the processing unit to perform the steps of the method according to claim 7.

15. Computer readable data carrier storing a computer program product according to claim 14.
